(19) United States
(12) Patent Application Publication
(10) Pub. No.: US 2016/0174810 A1
(43) Pub. Date: Jun. 23, 2016

(54) IMAGING DEVICES AND RELATED METHODS

(71) Applicant: Boston Scientific Scimed, Inc., Maple Grove, MN (US)

(72) Inventors: Russell P. DRESHER, Hudson, MA (US); Paul D. AQUILINO, Walpole, MA (US)

(73) Assignee: Boston Scientific Scimed, Inc., Maple Grove, MN (US)

(21) Appl. No.: 14/974,066

(22) Filed: Dec. 18, 2015

Related U.S. Application Data
(60) Provisional application No. 62/094,733, filed on Dec. 19, 2014.

(51) Int. Cl. A61B 1/00; A61B 1/06; A61B 1/05

(52) U.S. Cl. CPC A61B 1/00009; A61B 1/05; A61B 1/00045; A61B 1/00016; A61B 1/0676; A61B 1/00066

(57) ABSTRACT

According to aspects of the present disclosure, an imaging medical device may include an elongated member having a proximal end, and distal end having an imaging component configured to transmit an analog imaging signal. The assembly also may include a proximal member having a distal end connected to the proximal end of the elongated member. The proximal end may be configured to receive a degraded analog imaging signal based on the analog signal and transmit an amplified analog imaging signal based on the degraded analog imaging signal. The analog imaging signal transmitted by the imaging component may be degraded during transmission to the proximal member to become the degraded analog imaging signal. The assembly also may include a signal processor in electronic communication with the proximal member and configured to receive and process the amplified analog imaging signal and generate a digital signal based on the amplified analog signal.

### CROSS-REFERENCE TO RELATED APPLICATIONS

This application claims the benefit of U.S. Provisional Application No. 62/094,733, filed Dec. 19, 2014, the disclosure of which is incorporated herein by reference in its entirety.

### TECHNICAL FIELD

Various aspects of the present disclosure relate generally to medical systems and devices. In particular, exemplary embodiments relate to endoscopic medical devices for enhanced visualization. Embodiments of the present disclosure also cover methods of manufacturing and methods of using such systems and devices.

### BACKGROUND

Medical devices are often inserted into the body to perform a therapeutic or diagnostic procedure inside a patient's body. An example of such a device is an endoscope, which is a flexible instrument introduced into the body for diagnostic or therapeutic purposes. Typically, endoscopic devices are inserted into the body through an opening (a natural opening or an incision), and are delivered to a work site inside the body through a body channel, such as, for example, the esophagus. Imaging devices incorporated in endoscopes allow a surgeon to see the work site from outside the body and remotely operate the endoscope to perform a desired diagnostic/therapeutic procedure at the work site. There are many different types of endoscopes in use today, and embodiments of the current disclosure may be applied with any of these different types of endoscopes or other medical devices. In general, embodiments of the current disclosure may be applicable with any type of medical device that can be inserted into a body, and that allows a surgeon outside the body to visualize a region inside the body. For the sake of brevity, however, the novel aspects of the current disclosure will be described with reference to an endoscope.

In a typical application, a distal end of an endoscope may be inserted into the body through an opening in the body. This opening may be a natural anatomic opening, such as, for example, the mouth, rectum, vagina, etc., or an incision made on the body. The endoscope may be pushed into the body such that the distal end of the endoscope proceeds from the point of insertion to a region of interest (work site) within the body by traversing a body channel. The endoscope may include one or more lumens extending longitudinally from the proximal end to the distal end of the endoscope. These lumens may deliver various diagnostic/treatment devices from outside the body to the work site to assist in the performance of the intended procedure at the work site.

Among others, these lumens may include an imaging lumen that may include an imaging component to capture images of the work site and deliver the image outside the body for viewing. Endoscopic visualization is used to diagnose and/or treat any number of conditions in the gastric, pulmonary, and urologic tracts. Endoscopes are required to not only navigate to the target site, but also to provide adequate visualization for diagnosis and/or treatment. Adequate visualization requires sufficient image quality to represent details within the anatomy under observation.

Endoscopes may use a variety of signaling schemes and transmission lines to transfer the image data at the distal tip of the endoscope to a signal processor at or near a proximal portion of the endoscope. An analog image signal produced by the imaging component at the distal end of the endoscope is converted to a digital signal. Some endoscopes perform that digitizing conversion at the distal tip of the endoscope. One advantage of converting the analog signal to a digital signal at the distal tip of the endoscope is that the noise-immune digital signal may travel the length of the endoscope with little to no degradation of the image data, thus, potentially avoiding analog signal degradation during transmission along the length of the endoscope. However, an analog-to-digital conversion method at the distal tip may increase the size requirements of the device and also may add to the cost of the endoscope. Furthermore, for endoscopes designed to be used in small diameter anatomy, the added size of a distal end required to fit the digitization components may not be feasible. In these situations, the analog signal may be transmitted to the nearest location where digitization can occur. Often times, this means that the analog signal is transmitted from the distal tip of the endoscope to the signal processor.

For long transmission lines (e.g. in a long endoscope) the analog signal may be susceptible to degradation and noise as it travels the length of the endoscope. If the length of the endoscope cannot be shortened, the imaging component may be designed to drive a larger load (e.g. a stronger signal) to reduce the effects of degradation. However, driving a larger load may result in a larger imager, which may not be feasible for endoscopes used in small diameter anatomy.

### SUMMARY

Aspects of the present disclosure relate to, among other things, medical devices, and methods of imaging portions of the body.

According to aspects of the present disclosure, an imaging medical device may include an elongated member comprising a proximal end, and a distal end having an imaging component configured to transmit an analog imaging signal. The device also may include a proximal member having a distal end connected to the proximal end of the elongated member which may be configured to receive a degraded analog imaging signal based on the analog signal and transmit an amplified analog imaging signal based on the degraded analog imaging signal. The analog imaging signal transmitted by the imaging component may be degraded during transmission to the proximal member to become the degraded analog imaging signal. The device may include a signal processor in electronic communication with the proximal member and configured to receive and process the amplified analog imaging signal and generate a digital signal based on the amplified analog signal.

In addition or alternatively, the device may include one or more of the following features. A display in digital/electronic communication with the signal processor. The display may be configured to display a digital image based on the digital signal generated by the signal processor. The display may be in wireless digital communication with the signal processor. The elongated member may include a flexible distal portion configured to traverse a body lumen. The elongated member may include a plurality of lumens. The imaging component may have an imager and one or more illumination members configured to illuminate a portion of the body to be imaged by the imager. The proximal member may include a handle configured to control movement of the elongated member. The handle may have a signal amplification processor configured to process the degraded analog imaging signal and transmit the amplified analog imaging signal based on the degraded analog imaging signal to the signal processor. The imaging component may be configured to transmit the analog imaging signal to the proximal member via low voltage differential signal (LVDS). A sensor may be configured to detect the degraded analog signal and/or the amount of degradation. The amplified analog imaging signal may be substantially the same as the analog imaging signal generated and transmitted by the imaging component. A plurality of processors may be spaced along the proximal member, each processor may be configured to amplify the degraded analog imaging signal. A voltage of a first amplified analog imaging signal generated by a first processor at a proximal end of the proximal member may be greater than a voltage of a second amplified analog imaging signal generated by a second processor distal to the first processor.

According to aspects of the present disclosure, an imaging medical device may include an elongated member comprising a proximal end, and a distal end having an imaging component configured to transmit an analog imaging signal. The device may include a handle having a distal end connected to the proximal end of the elongated member, which may be configured to process the analog imaging signal transmitted by the imaging component and transmit a digital imaging signal based on the processed analog imaging signal. The device also may include a signal processor in digital communication with the handle and configured receive the digital imaging signal.

In addition or alternatively, the device may include one or more of the following features. A display in electronic communication with the signal processor. The display may be configured to display a digital image based on the digital signal. The display may be in wireless digital communication with the signal processor.

According to aspects of the present disclosure, a method of transferring image data may include, transmitting an analog signal from an imaging component in a distal portion of a medical device, receiving, in a proximal portion of the medical device, a degraded analog signal that is a degraded form of the transmitted analog signal, generating, at the proximal portion of the medical device, a conditioned analog signal based on the degraded analog signal, wherein the conditioned analog signal is approximately the same as the analog signal transmitted by the imaging component; transmitting, to an external controller, the conditioned analog signal for processing the conditioned analog signal; generating a digital signal; and displaying an image based on the digital signal.

In addition or alternatively, the method may include one or more of the following features. The analog signal transmitted from the imaging component may be transmitted via a low voltage differential signal. The method may further include generating, at a position between the distal portion and the proximal portion of the medical device, an intermediately conditioned analog signal based on the degraded analog signal, wherein a voltage of the intermediately conditioned analog signal may be less than a voltage of the analog signal transmitted by the imaging component. The image may be displayed on a display in wireless communication with the external controller. The imaging component may include one or more illumination members which may be configured to illuminate portion of the body to be imaged by the imaging component.

According to aspects of the present disclosure, an imaging system may include a display and a medical device, the medical device may include: an elongated member comprising a proximal end and a distal end having an imaging component configured to transmit an analog signal; a handle having a distal end connected to the proximal end of the elongated member and configured to process the analog imaging signal transmitted by the imaging component and transmit a digital imaging signal based on the processed analog imaging signal, and a signal processor in digital communication with the handle and configured to receive the digital imaging signal.

It may be understood that both the foregoing general description and the following detailed description are exemplary and explanatory only and are not restrictive of the claimed features.

### BRIEF DESCRIPTION OF THE DRAWINGS

The accompanying drawings, which are incorporated in and constitute a part of this specification, illustrate exemplary aspects of the present disclosure and together with the description, serve to explain the principles of the disclosure.

FIG. 1 is a schematic view of an embodiment of a medical imaging system.

FIG. 2 is a block diagram of an exemplary method for transferring medical image data, according to an exemplary embodiment of the present disclosure.

FIG. 3 is a block diagram of an exemplary method for transferring medical image data, according to another exemplary embodiment of the present disclosure.

### DETAILED DESCRIPTION

Overview

The present disclosure is drawn to medical devices and methods of using medical devices. Portions of the medical device may be used to capture images of a patient's body during a medical procedure. The medical device also may include components to process signals from an imaging component of the medical device to provide a digital image.

Reference will now be made in detail to aspects of the present disclosure, examples of which are illustrated in the accompanying drawings. Wherever possible, the same reference numbers will be used throughout the drawings to refer to the same or like parts. The term “distal” refers to a portion farthest away from a user when introducing a device into a subject. By contrast, the term “proximal” refers to a portion closest to the user when placing the device into the subject.

Exemplary Aspects

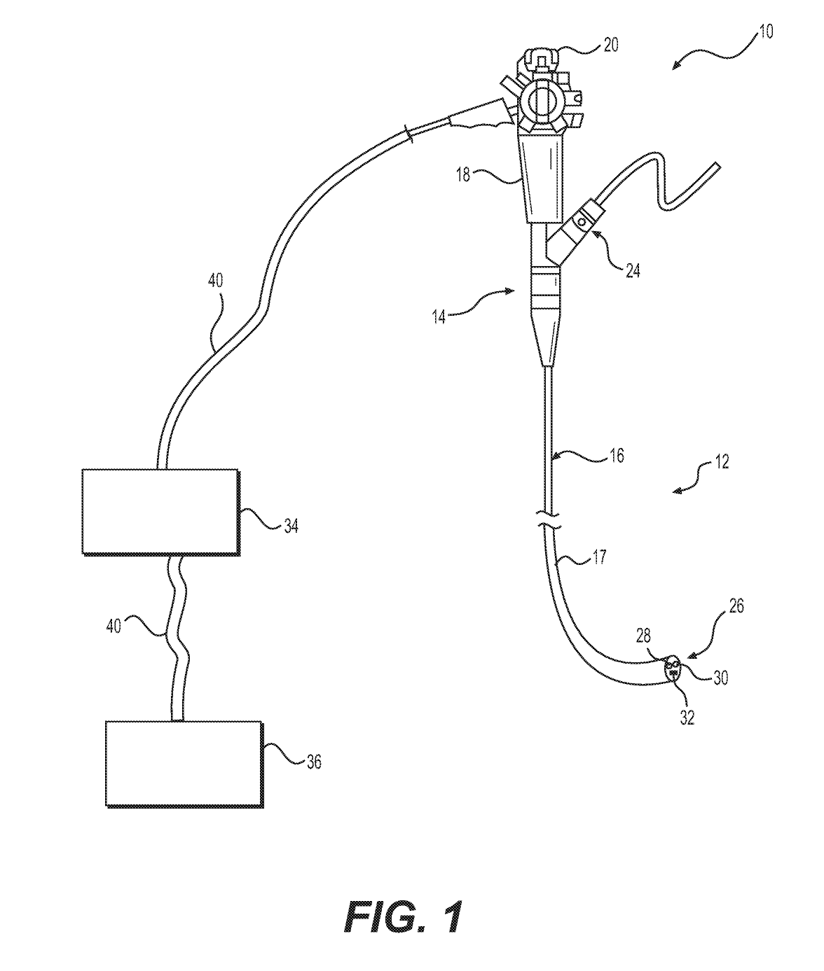

FIG. 1 illustrates a schematic view of an embodiment of a medical imaging system 10. The system 10 may include a medical device 12 having a proximal portion 14 and a distal portion 16 for insertion in the body. The proximal portion 14 may include a handle 18 having a controller 20 configured to control various properties of the device 12. For example, the controller 20 may be configured to control the actuation and movement of the distal portion 16 of the device 12 in any suitable manner, such as via mechanical (e.g. wires, cams, pulleys), electromechanical, and/or electrical mechanisms. The proximal portion 14 also may include an introducer portion 24 configured to provide an entry for the insertion of one or more medical tools through the device 12 and out of a distal end 26 of the medical device 12. For example, graspers, ablators, cutting tools, and/or imaging tools may be inserted via the introducer 24, and out of the distal end 26 of the medical device 12.

The distal portion 16 of the device 12 may include an elongated member 17 connected to the handle 18 and having one or more lumens 28, 30. The elongated member 17 may have any suitable size and shape for insertion in the body. The elongated member 17 may be flexible and have various suitable surface properties. The tool(s) inserted via the introducer 24 at the proximal portion 14 of the device 12 may be inserted through the various lumens 28, 30 extending through the elongated member 17 and out of the distal end 26 of the device 12, to a target site in the body. The lumens 28, 30 may have any suitable size, shape, and properties to accommodate the inserted tools.

As shown in FIG. 1, the distal end 26 of the device 12 also may include an imaging component 32 which may be inserted through the elongated member 17, for example via the introducer 24. Alternatively, the imaging component 32 may be formed as a part of the medical device 12 and may be adjustably movable or may be stationary within the elongated member 17. The imaging component 32 may be positioned at any suitable position along the elongated member 17, for example aligned with the distal end 26 of the elongate member, or proximate (e.g. distal to or proximal to) the distal end 26 of the elongated member 17.

The imaging component 32 may include various parts configured to capture images, such as light sources (e.g. LEDs), lenses, circuitry, digital memory, power supply (e.g. batteries), and/or processors. The imaging component 32 may be controlled in any suitable manner, such as electronically controlled via controller 20. The imaging component 32 may be configured to capture images in any suitable manner. For example, the imaging component 32 may capture images as analog image signals. The analog image signals may include a low-voltage signal containing the intensity information for each line in an image frame in combination with timing information to provide a synchronized display. The analog signal for a single horizontal video line may include a horizontal sync signal, back porch, active pixel region, and front porch, etc. The analog signal may be transmitted to one or more processors within the device 12, for example in the distal portion 16 and/or the proximal portion 14 in any suitable manner. For example, the analog signal from the imaging component 32 may be transmitted to the one or more processing units via one or more wires along the device 12. The one or more processing units may condition and/or convert the analog signal into a digital signal in any suitable manner, for example, via the methods 200 and 300 described below or a combination of the methods 200 and 300. In some examples, the analog signal may be differential or single-ended.

The proximal portion 14 and distal portion 16 of the device 12 may include various components configured to control the movement and other characteristics of the device 12. For example, one or more electronic circuits may be disposed in the device 12 for controlling the medical device 12 and/or the tools inserted through the device 12.

The system 10 also may include one or more signal processing control units 34 in electronic communication with the device 12 either wirelessly or via wires 40. The one or more control units 34 may include electronic circuitry configured to receive, process, and/or transmit data and signals between the device 12 and other devices. For example, the control unit 34 may be in electronic communication with a display 36 configured to display images based on image data and/or signals processed by the control units 34, which may have been generated by the imaging component 32 of the device 12. The control unit 34 may be in electronic communication with the display 36 in any suitable manner, either via wires 40 or wirelessly.

The display 36 may be manufactured in any suitable manner and may include touch screen inputs and/or be connected to various input and output devices such as, mouse, electronic stylus, printers, servers, and/or other electronic portable devices.

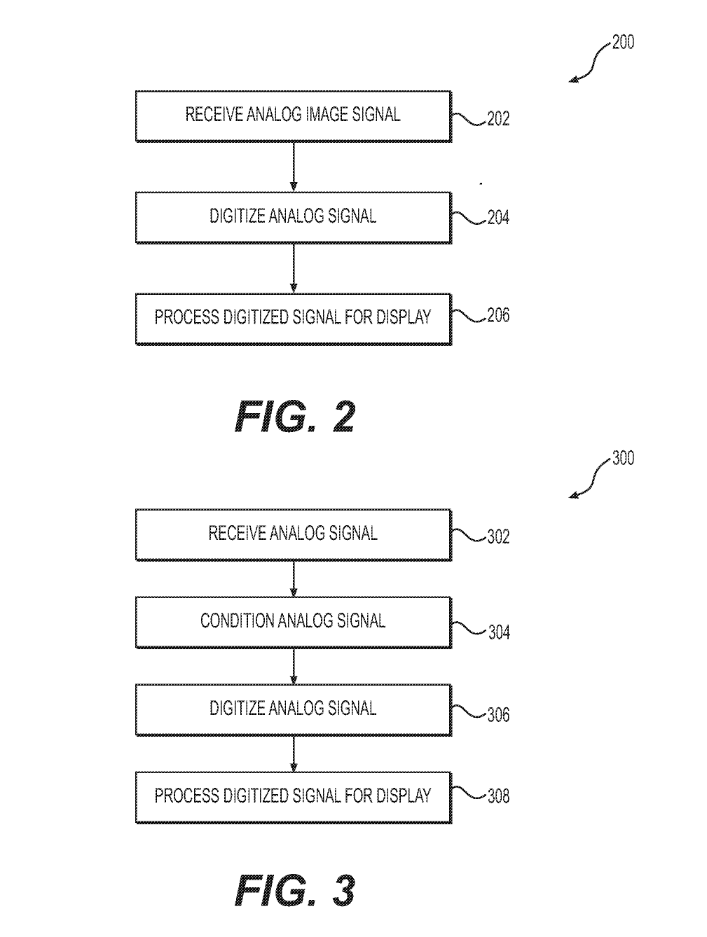

The method 200 shown in FIG. 2 is an example of a method of displaying an image from an imaging component, such as imaging component 32 of device 12. The method 200 may include a step 202 of receiving an analog image signal from the imaging component 32. The analog signal may be transmitted via one or more wires manufactured using any suitable material(s) configured to transmit electrical signals. The analog signals may be received by one or more processing units having suitable circuitry components (e.g. processors, memory, and, power supply) and programs saved in the memory, for converting the analog signals into digital signals in any suitable manner at step 204. For example, the analog signals may be converted to digital signals by one or more digitizing processors configured to execute digitizing program instructions. The digitizing processor(s) may be positioned at any suitable location in the device 12, for example in the handle 18, or along the elongated member 17 (e.g. in close proximity to the imaging component 32). An advantage of having the digitizing processing in the handle is that the handle has sufficient size to accommodate the necessary processing, and the elongate member cross-sectional area may be minimized since it does not need to accommodate such processing. In some example, the digitizing processor(s) may be movable along the medical device. In examples in which the digitizing processors are positioned close (e.g. adjacent to) the imaging component 32, the analog signal received from the imaging component 32 and by the digitizing processor may be equal to the analog imaging signal sent by the imaging component 32, e.g. the analog signal may not have suffered any degradation.

In addition, the digitizing processor may include any suitable support circuitry such as voltage regulators and/or electrostatic discharge (ESD) components configured to provide protection from electrostatic discharges. The support circuitry may be integrated on the same printed circuit board (PCB) or integrated circuit (IC) as the digitizing processor.

In some examples, (e.g. where the digitizing processor is not positioned close to the imaging component 32) one or more conditioning processors may be configured to amplify any degradation in the analog signal from the imaging component 32 and transmit the conditioned analog signal to the digitizing processor. A degraded signal may be any signal having a signal strength less than the signal generated by the imaging component 32. The degraded analog signal may be amplified in any suitable manner, for example, by circuitry having transistor-transistor logic (TTL). In some examples, the amplification and digitization of the analog signal may be provided by the same processor. The conditioning unit processor(s) may amplify any degradation in the original analog image signal from the imaging component 32 in any suitable manner. The digital image signal based on the analog image signal may then by further processed at step 206 either by the digitizing processor or an external control unit, such as control unit 34 for displaying images on a display, such as display 36.

In some examples, the digitizing and/or the conditioning processors may include a sensor configured to detect any degradation in the analog signal in any suitable manner, for example, the analog image signal may be compared to the signal received by the digitizing processor, and if the analog imaging signal is not the same (e.g. less than) the analog imaging signal from the imaging component 32, or below a pre-determined threshold value (e.g. electronically pre-determined and/or manually programmed), then it may be determined that the analog imaging signal has been degraded during transmission and should be conditioned prior to being digitized. Alternatively, if it is determined by the sensor(s) that the analog imaging signal has been degraded, it may be determined that the digitizing processor should be moved closer to the imaging component 32 in order avoid degradation. In such examples, an optimized distance between the imaging component 32 and the digitizing processor may be electronically determined via one or more processors and saved in memory (e.g. memory in the handle 18) for retrieval and/or further processing.

Alternatively, the method 300 as shown in FIG. 3 may be used to provide a digital image from a medical device. The method 300 may include a step 302 of receiving an analog signal 302 from an imaging component, such as imaging component 32. One or more conditioning processors may be configured to amplify any degradation in the analog signal from the imaging component 32 at step 304. The conditioning processors may be part of a circuit having any suitable components, such as memory and power supply. The memory may include program instructions for conditioning analog signals, and the conditioning processor may be configured to execute the conditioning program instructions to condition (e.g. amplify) the analog imaging signals.

The one or more conditioning processors may be positioned at any suitable location(s) in the device 12, for example in the handle 18, or along the elongated member 17. An advantage of having the digitizing processing in the handle or a proximal portion of the device 12 is that these portions of the device 12 may have sufficient size to accommodate the necessary processing, and the elongate member cross-sectional area may be minimized since it does not need to accommodate such processing. The conditioned analog signal may be substantially/approximately equal to the original analog signal transmitted by the imaging component 32 (e.g. within 10% of the signal value generated by the imaging component). In some examples, the method may include a step of determining (e.g. via sensor(s)) if the analog signal has been degraded relative to the original analog signal transmitted by the imaging component 32, and determining the extent of the signal degradation. The determination of any degradation may be provided by the conditioning processor and/or a separate sensor in the medical device configured to detect analog signal strength.

The conditioned analog signal may be transmitted by the conditioning processor outside the device 12 to a control unit 34, which may include a digitizing processor configured to convert the conditioned analog signal to a digital signal at step 306. The digital signal may then be processed for display at step 308. For example, the original analog signal transmitted by the imaging component 32 may have a value of about 1.0V and may be degraded to about 0.6 V during transmission along the device 12. A first conditioning processor may be positioned proximal to the imaging component, in the elongated member 17. The first conditioning processor may amplify the degraded analog signal of 0.6 V to a value of about 0.8 V. A second conditioning processor may be positioned proximal to the first conditioning processor (e.g. in the handle 18) and may further condition the analog signal received from the first conditioning processor (of 0.8V) and transmit an amplified analog signal of about 1.0 V for digitization and subsequent display of the image.

The device 12 and/or the control unit 34 may connect to a platform or a server. The platform or server or the like may include a data communication interface for packet data communication, such as for transmission of analog and/or digital imaging signals. The platform also may include a central processing unit (CPU) in the form of one or more processors, for executing program instructions. The platform may include an internal communication bus program storage and data storage for various data files to be processed and/or communicated by the platform such as ROM and RAM, although the server often receives programming and data via network communications. The hardware elements, operating systems, and programming languages of such equipment are conventional in nature, and it is presumed that those skilled in the art are adequately familiar therewith. The server also may include input and output ports to connect with input and output devices such as keyboards, mice, touchscreens, monitors, displays (e.g. display 36), etc. Of course, the various server functions may be implemented in a distributed fashion on a number of similar platforms, to distribute the processing load. Alternatively, the servers may be implemented by appropriate programming of one computer hardware platform.

Program aspects of the technology may be thought of as “products” or “articles of manufacture” typically in the form of executable code and/or associated data that is carried on or embodied in a type of machine-readable medium (e.g. programming configured to condition and/or digitize analog imaging signals). “Storage” type media include any or all of the tangible memory of the computers, processors or the like, or associated modules thereof, such as various semiconductor memories, tape drives, disk drives and the like, which may provide non-transitory storage at any time for the software programming.

All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links.

The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

The disclosed medical devices and methods may be utilized in any suitable application involving illumination and/or visualization in the body during a therapeutic and/or diagnostic medical procedure. Any aspect set forth herein may be used with any other aspect set forth herein. The devices may be used in any suitable medical procedure, may be advanced through any suitable body lumen and body cavity. For example, the devices described herein may be used through any natural body lumen or tract, including those accessed orally, vaginally, rectally, nasally, urethrally, or through incisions in any suitable tissue.

It will be apparent to those skilled in the art that various modifications and variations can be made in the disclosed medical devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. An imaging medical device comprising:
an elongated member comprising a proximal end, and distal end having an imaging component configured to transmit an analog imaging signal;
a proximal member having a distal end connected to the proximal end of the elongated member and configured to receive a degraded analog imaging signal based on the analog signal and transmit an amplified analog imaging signal based on the degraded analog imaging signal, wherein the analog imaging signal transmitted by the imaging component is degraded during transmission to the proximal member to become the degraded analog imaging signal; and
a signal processor in electronic communication with the proximal member and configured to receive and process the amplified analog imaging signal and generate a digital signal based on the amplified analog signal.

2. The device of claim 1, further comprising a display in electronic communication with the signal processor, wherein the display is configured to display a digital image based on the digital signal generated by the signal processor.

3. The device of claim 2, wherein the display is in wireless digital communication with the signal processor.

4. The device of claim 1, wherein the elongated member comprises a flexible distal portion configured to traverse a body lumen.

5. The device of claim 1, wherein the elongated member comprises a plurality of lumens.

6. The device of claim 1, wherein the imaging component comprises an imager, and one or more illumination members configured to illuminate a portion of the body to be imaged by the imager.

7. The device of claim 1, wherein the proximal member comprises a handle configured to control movement of the elongated member.

8. The device of claim 7, wherein the handle comprises a signal amplification processor configured to process the degraded analog imaging signal and transmit the amplified analog imaging signal based on the degraded analog imaging signal to the signal processor.

9. The device of claim 1, wherein the imaging component is configured to transmit the analog imaging signal to the proximal member via low voltage differential signally (LVDS).

10. The device of claim 1, further comprising a sensor configured to detect the degraded analog signal.

11. The device of claim 1, wherein the amplified analog imaging signal is substantially the same as the analog imaging signal generated and transmitted by the imaging component.

12. The device of claim 11, further comprising a plurality of processors spaced along the proximal member, wherein each processor is configured to amplify the degraded analog imaging signal.

13. An imaging medical device comprising:
an elongated member comprising a proximal end and a distal end having an imaging component configured to transmit an analog imaging;
a handle having a distal end connected to the proximal end of the elongated member and configured to process the analog imaging signal transmitted by the imaging component and transmit a digital imaging signal based on the processed analog imaging signal, and
a signal processor in digital communication with the handle and configured receive the digital imaging signal.

14. The device of claim 13, further comprising a display in digital communication with the signal processor, wherein the display is configured to display a digital image based on the digital signal.

15. The device of claim 14, wherein the display is in wireless digital communication with the signal processor.

16. A method of transferring image data comprising:
transmitting an analog signal from an imaging component in a distal portion of a medical device;
receiving, in a proximal portion of the medical device, a degraded analog signal that is a degraded form of the transmitted analog signal,
generating, at the proximal portion of the medical device, a conditioned analog signal based on the degraded analog signal, wherein the conditioned analog signal is approximately the same as the analog signal transmitted by the imaging component;
transmitting, to an external controller, the conditioned analog signal for processing the conditioned analog signal;
generating a digital signal; and
displaying an image based on the digital signal.

17. The method of claim 16, wherein the analog signal transmitted from the imaging component is transmitted via a low voltage differential signal.

18. The method of claim of claim 16, further comprising generating, at a position between the distal portion and the proximal portion of the medical device, an intermediately conditioned analog signal based on the degraded analog signal, wherein the intermediately conditioned analog signal has a voltage that is less than a voltage of the analog signal transmitted by the imaging component.

19. The method of claim 16, wherein the image is displayed on a display in wireless communication with the external controller.

20. The method of claim 16, wherein the imaging component comprises an imager, and one or more illumination members configured to illuminate a portion of the body to be imaged by the imager
